(19) United States
(12) Patent Application Publication
(10) Pub. No.: US 2010/0178293 A1
(43) Pub. Date: Jul. 15, 2010

(54) USE OF ANTIBODIES AGAINST THE CD52 ANTIGEN FOR THE TREATMENT OF NEUROLOGICAL DISORDERS, PARTICULARLY TRANSMISSIBLE SPONGIFORM ENCEPHALOPATHY AND ALZHEIMER'S DISEASE

(76) Inventor: Olaf Weber, Wulfrath (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/665,401

(22) PCT Filed: Jun. 10, 2008
(86) PCT No.: PCT/EP08/04609

§ 371 Date: Dec. 18, 2009

(30) Foreign Application Priority Data
Jun. 22, 2007 (EP) 07012262.7

(51) Int. Cl. A61K 39/395; A61P 25/28; C07K 16/00

(52) U.S. Cl. 424/134.1; 424/139.1; 530/387.3; 530/388.1; 530/387.1

(57) ABSTRACT

The present invention relates to the use of antibodies against the CD52 antigen, specifically monoclonal antibodies, for the production of medicaments for the treatment of diseases of the central nervous system, in particular transmissible spongiform encephalopathies, diseases that are also called prion diseases.

The present invention relates to the use of antibodies against the CD52 antigen, specifically monoclonal antibodies, for the production of medicaments for the treatment of diseases of the central nervous system, in particular transmissible spongiform encephalopathies, diseases that are also called prion diseases. These diseases occur in humans and animals and are due to conformational conversion of PrP(C), a cellular glycoprotein of unknown function, into PrP(Sc), an isoform that appears to be infectious in the absence of nucleic acids. The diseases are either genetic or infectious. Although prions are most efficiently transferred by intracerebral inoculation, peripheral administration has caused kuru, iatrogenic Creutzfeldt-Jakob disease (CJD), bovine spongiform encephalitis (BSE), and new variant CJD. Neurological disease after peripheral inoculation depends on prion expansion within cells of the lymphoreticular system (LRS) (Lasmezas C I, Cesbron J Y, Deslys J P, Demaimay R, Adjou K T, Rioux R, Lemaire C, Locht C, Dormont D. (1996) Immune system-dependent and -independent replication of the scrapie agent. J. Virol. 70 (2):1292-5). Klein et al. have shown that the reticuloendothelial system, particularly differentiated B cells appear to play a crucial role in neuroinvasion of scrapie regardless of B-cell receptor specificity (Klein M A, Frigg R, Flechsig E, Raeber A J, Kalinke U, Bluethmann H, Bootz F, Suter M, Zinkernagel R M, Aguzzi A. (1997) A crucial role for B cells in neuroinvasive scrapie. Nature. 390(6661):662-3).

Prion diseases are thought to be not treatable by conventional medicines. Prevention, if possible, was the only strategy to combat the disease. Progress has been made in recent years, demonstrating principal reversibility of the neuropathological features and protection from clinical symptoms in animal models and introducing potential pharmaceutical agents. Among the most promising ones, antibodies have been shown to be protective against prion disease and heterocyclic small-molecule compounds are investigated as compounds in clinical trials (Korth C, Peters P J (2006) Emerging pharmacotherapies for Creutzfeldt-Jacob disease. Arch Neurol 63 (4):497-501). However, to date no agents are close to being used in treatment of the disease in humans.

Alzheimer's disease (AD) is the most common dementing illness and is pathologically characterized by deposition of amyloid-beta as senile plaques.

Although the role of the immune system in the pathogenesis of AD is still not fully understood it is documented that brain inflammatory mechanisms mediated by reactive glia are activated in response to the amyloid plaques. In addition, reports suggest that T-cells are activated in AD patients, and that these cells exist both in the periphery and as infiltrates in the brain (Town T, Tan J, Flavell R A, Mullan M. (2005). T-cells in Alzheimer's disease. Neuromolecular Med. 7(3):255-64). In line with this evidence it was demonstrated that Ibuprofen suppresses plaque pathology and inflammation in a mouse model for Alzheimer's disease (Lim G P, Yang F, Chu T, Chen P, Beech W, Teter B, Tran T, Ubeda O, Ashe K H, Frautschy S A, Cole G M. (2000) Ibuprofen suppresses plaque pathology and inflammation in a mouse model for Alzheimer's disease. J. Neurosci. 20(15):5709-14). Today, there exists no satisfying treatment against this devastating disease.

As is clear from the abovementioned prior art, no therapeutic method has so far been disclosed which allows to treat patients suffering from Alzheimer's disease or transmissible spongiform encephalopathies effectively. This therapeutic method should be superior to current therapies. This problem has been solved by the current invention by providing pharmaceutical compositions for the treatment of Alzheimer' s disease or transmissible spongiform encephalopathies (TSE) which contain antibodies against CD52, more preferably monoclonal antibodies against CD52, or most preferably alemtuzumab (Campath, Bayer Schering Pharma AG, a humanized monoclonal antibody against CD52).

Thus, the present invention relates to:
1. The use of antibodies against CD52 for the preparation of medicaments for the treatment of neurological diseases, particularly Alzheimer Disease (AD) and transmissible spongiform encephalopaties (TSE). According to the invention antibody against CD 52 is understood to be either a murine, a chimeric, a humanized or a fully human monoclonal antibody. The latter could be produced using a Human Combinatorial Antibody Library.
2. The invention also relates to the use of antibodies against CD52 for the preparation of medicaments for the treatment of neurological diseases, particularly Alzheimer Disease (AD) and transmissible spongiform encephalopathy (TSE) in combination with substances which are effective form support therapy for the production of medicaments against TSE in humans and animals and AD in humans.

The pharmaceutical composition of the present invention may be administered in oral forms, such as, without limitation normal and enteric coated tablets, capsules, pills, powders, granules, elixirs, tinctures, solution, suspensions, syrups, solid and liquid aerosols and emulsions. They may also be administered in parental forms, such as, without limitation, inravenous, intraperitoneal, subcutaneous, intramuscular and the like forms well-know to those of ordinary skill in the pharmaceutical arts. The pharmaceutical composition of the present invention can be administered by means of implanted pumps that release the composition in a controlled manner. The pharmaceutical composition of the present invention can be administered in intranasal form via topical use of suitable intranasal vehicles, or via transdermal routes, using transdermal delivery systems well-known to those of ordinary skilled in the art.

The dosage regimen with the use of the pharmaceutical composition of the present invention is selected by one of ordinary skill in the arts, in view of a variety of factors, including, without limitation, age, weight, sex, and medical condition of the recipient, the severity of the condition to be treated, the route of administration, the level of metabolic and excretory function of the recipient, the dosage form employed.

The pharmaceutical compositions of the present invention are preferably formulated prior to administration and include one or more pharmaceutically acceptable excipients. Excipients are inert substances such as, without limitations, carriers, diluents, flavoring agents, sweeteners, lubricants, solubilizers, suspending agents, binders, tablet disintegrating agents and encapsulating material.

The formulation may be in unit dosage form, which is a physically discrete unit containing a unit dose, suitable for administration in human or other mammals. A unit dosage form can be a capsule or tablets, or a number of capsules or tablets. A “unit dose” s a predetermined quantity of the active pharmaceutical composition of the present invention, calculated to produce the desired therapeutic effect, in association with one or more excipients. Dosages will vary from about 100 μg to about 200 mg per application or will be based on mg/kg/day.

The pharmaceutical compositions of the present invention may be administered in a single daily dose, or the total daily dose may be administered in divided doses, two, three, or more times per day. Where delivery is via transdermal forms, of course, administration is preferably continuous.

The treatment schedule may vary from once a day over a defined period of time (preferably from 1 day to 1000 days) up to once a year over lifetime.

Surprisingly, antibodies against CD52 (Campath (Bayer Schering Pharma AG), alemtuzumab; a humanized monoclonal antibody directed to CD52), can inhibit the spread of prions in an animal model (Example 1). Moreover, Campath is active in a mouse model of Alzheimer' s disease (Example 2).

In particular, the invention relates to:
1. The use of an antibody against CD 52 for the production of a medicament for treating a neurological disease.
2. The use according to count 1, wherein the antibody is a monoclonal antibody.
3. The use according to count 2, wherein the antibody is a humanized monoclonal antibody.
4. The use according to count 2, wherein the antibody is alemtuzumab.
5. The use according to counts 1-4, wherein the neurological disease is a transmissible spongiform encephalopathy.
6. The use according to counts 1-4, wherein the neurological disease is Alzheimer's disease.
7. The use according to counts 1-6, wherein at least one additional neurologically active agent is used.
8. The use according to counts 1-6, wherein an analgesic is used as a further therapeutic agent.
9. The use according to count 8, wherein a further therapeutic agent selected from the group consisting of ibuprufene, acetylsalicylic acid, naproxene, acetaminophene, Cox-2 inhibitors), antidepressants such as without limitation selective serotonine inhibitors or tricyclic antidepressants, anticonvulsants, capsicain, and mexiletine.
10. A pharmaceutical composition, comprising alemtuzumap and a therapeutic agent selected from the group consisting of ibuprufene, acetylsalicylic acid, naproxene, acetaminophene, Cox-2 inhibitors), antidepressants such as without limitation selective serotonine inhibitors or tricyclic antidepressants, anticonvulsants, capsicain, and mexiletine.
11. The pharmaceutical composition according to count 10 for the treatment of neurological diseases.
12. The pharmaceutical composition according to count 11, wherein the neurological disease is selected from group consisting of Alzheimer's disease and a transmissible spongiform encephalopathy.
13. A pharmaceutical composition comprising an antibody against CD 52 and a further therapeutic agent.
14. A pharmaceutical composition comprising a monoclonal antibody against CD 52 and a further therapeutic agent.
15. A pharmaceutical composition according to count 13 or 14, wherein the further therapeutic agent is selected from the group of further neurologically active agents or analgesic agents such as ibuprufene, acetylsalicylic acid, naproxene, acetaminophene, Cox-2 inhibitors), antidepressants such as without limitation selective serotonine inhibitors or tricyclic antidepressants, anticonvulsants, capsicain, and mexiletine.
16. A pharmaceutical composition according to count 14 or 15, wherein the monoclonal antibody is alemtuzumap.
17. A pharmaceutical composition comprising an antibody against CD 52 for treating a neurological disease.
18. A pharmaceutical composition according to count 17, wherein the antibody is a monoclonal antibody.
19. A pharmaceutical composition according to count 18, wherein the antibody is a humanized monoclonal antibody.
20. A pharmaceutical composition according to count 19, wherein the antibody is alemtuzumab.
21. A pharmaceutical composition according to counts 17-20, wherein the neurological disease is a transmissible spongiform encephalopathy.
22. A pharmaceutical composition according to counts 17-20, wherein the neurological disease is Alzheimer's disease.
23. A pharmaceutical composition according to counts 17-22, wherein at least one additional neurologically active agent is used.
24. A pharmaceutical composition according to counts 17-22, wherein an analgesic is used as a further therapeutic agent.
25. A pharmaceutical composition according to count 24, wherein a further therapeutic agent selected from the group consisting of ibuprofene, acetylsalicylic acid, naproxene, acetaminophene, Cox-2 inhibitors), antidepressants such as without limitation selective serotonine inhibitors or tricyclic antidepressants, anticonvulsants, capsicain, and mexiletine.

### EXAMPLES

### Example 1

RML, a mouse-adapted scrapie isolate is passaged in Swiss CD-1 mice. Inocula are 10% (w/v) homogenates of RML-infected CD-1 mouse brains in 0.32 M sucrose. Mice are infected i.p. with 100 μl of a 10-fold dilution of the inoculum in phosphate-buffered saline (PBS) containing 5% bovine serum albumin (BSA).

The following groups (n=10 animals/group) are treated as described:

| | Inoculation | Treatment | Application mode |
|---|---|---|---|
| Group 1 | RML prion | Campath | 100 μg i.v. 0 h after inoculation |
| Group 3 | RML prion | Campath | 100 μg i.v. 0 h and weekly for 4 |
| | | | weeks |
| Group 4 | RML prion | Saline | 100 μg i.v. 0 h after inoculation |
| Group 6 | RML prion | Saline | 100 μg i.v. 0 h and weekly for 4 |
| | | | weeks |
| Group 7 | Saline | Campath | 100 μg i.v. 0 h after inoculation |
| Group 9 | Saline | Campath | 100 μg i.v. 0 h and weekly for 4 |
| | | | weeks |
| Group 10 | Brain | Campath | 100 μg i.v. 0 h after inoculation |
| | homogenate | | |
| | of uninfected | | |
| | mouse | | |
| Group 12 | Brain | Campath | 100 μg i.v. 0 h and weekly for 4 |
| | homogenate | | weeks |
| | of uninfected | | |
| | mouse | | |

All animals are observed for approximately 1 year and scored daily for clinical signs of disease. Scrapie in mice is characterised by ataxia of gait, tremor, difficulty righting from a supine position, and rigidity in the tail. Occurrence of two of these four symptoms is used as the endpoint criterion for establishing a clinical diagnosis of scrapie. Western blots of brain homogenates are done to confirm the diagnosis. All results are analysed with Student's t test and Wilcoxon's two-sample rank-sum test.

### Example 2

Ten-month-old male and female Tg2576 Tg+ and Tg− are used (n=10 animals/group). Tg+ mice are treated with Campath 100 μg i.v. weekly for 6 months or placebo (saline), respectively before being sacrificed. TG− mice served as controls Animals are perfused before brain dissection with 0.9% normal saline followed by HEPES buffer, pH 7.2, containing protease inhibitors. Brain regions are dissected from one hemisphere and analyzed histologically and biochemically for the occurrence of amyloid plaques (methods compare to Lim G P, Yang F, Chu T, Chen P, Beech W, Teter B, Tran T, Ubeda O, Ashe K H, Frautschy S A, Cole G M. (2000) Ibuprofen suppresses plaque pathology and inflammation in a mouse model for Alzheimer's disease. J. Neurosci. 20(15):5709-14). Tg+ mice fed chow containing ibuprofen for 6 months serve as positive treatment control.

| | strain | Treatment | Application mode |
|---|---|---|---|
| Group 1 | Tg+ | campath | 100 μg i.v weekly for 6 months |
| Group 2 | Tg+ | Saline | i.v weekly for 6 months |
| Group 3 | Tg+ | Ibuprofen | 375 ppm feeding over 6 months |
| Group 1 | Tg− | campath | 100 μg i.v weekly for 6 months |
| Group 2 | Tg− | Saline | i.v weekly for 6 months |
| Group 3 | Tg− | Ibuprofen | 375 ppm feeding over 6 months |

1. The use of an antibody against CD 52 for the production of a medicament for treating a neurological disease.

2. The use according to claim 1, wherein the antibody is a monoclonal antibody.

3. The use according to claim 2, wherein the antibody is a humanized monoclonal antibody.

4. The use according to claim 2, wherein the antibody is alemtuzumab.

5. The use according to claim 1, wherein the neurological disease is a transmissible spongiform encephalopathy.

6. The use according to claim 1, wherein the neurological disease is Alzheimer' s disease.

7. The use according to claim 1, wherein at least one additional neurologically active agent is used.

8. The use according to claim 1, wherein an analgesic is used as a further therapeutic agent.

9. The use according to claim 8, wherein a further therapeutic agent selected from the group consisting of ibuprufene, acetylsalicylic acid, naproxene, acetaminophene, Cox-2 inhibitors, antidepressants, selective serotonine inhibitors, tricyclic antidepressants, anticonvulsants, capsicain, and mexiletine.

10. A pharmaceutical composition, comprising alemtuzumap and a therapeutic agent selected from the group consisting of ibuprufene, acetylsalicylic acid, naproxene, acetaminophene, Cox-2 inhibitors, antidepressants, selective serotonine inhibitors, tricyclic antidepressants, anticonvulsants, capsicain, and mexiletine.

11. The pharmaceutical composition according to claim 10 for the treatment of neurological diseases.

12. The pharmaceutical composition according to claim 11, wherein the neurological disease is selected from group consisting of Alzheimer's disease and a transmissible spongiform encephalopathy.

13. A method of using an antibody against CD 52, said method comprising preparing
a medicament for treating a neurological disease.
